(19) United States
(12) Patent Application Publication
(10) Pub. No.: US 2026/0263028 A1
(43) Pub. Date: Sep. 10, 2026

(54) ELECTRONIC DIGITAL STETHOSCOPE AND USES THEREOF

(71) Applicants: CHILDREN'S NATIONAL MEDICAL CENTER, Washington, DC (US); AUSCULTECH DX, LLC, Silver Spring, MD (US)

(72) Inventors: Raj SHEKHAR, Dayton, MD (US); Robin DOROSHOW, Bethesda, MD (US); Ravi AMBATI, New York, NY (US); Eric WELCH, Berkeley, CA (US); Youness ARJOUNE, Washington, DC (US); Tyler SALVADOR, Silver Spring, MD (US); Titus JOHN, Morrisville, NC (US)

(73) Assignees: CHILDREN'S NATIONAL MEDICAL CENTER, Washington, DC (US); AUSCULTECH DX, LLC, Silver Spring, MD (US)

(21) Appl. No.: 19/473,384

(22) PCT Filed: Apr. 17, 2024
(86) PCT No.: PCT/US2024/024919

§ 371 Date: Oct. 7, 2025

Related U.S. Application Data
(60) Provisional application No. 63/496,444, filed on Apr. 17, 2023.

(51) Int. Cl. A61B 7/04; H04R 1/46; H04R 3/04; H04R 7/00

(52) U.S. Cl. CPC A61B 7/04; H04R 1/46; H04R 3/04; H04R 7/00; H04R 2410/05

(57) ABSTRACT

A digital stethoscope, including a chest piece enclosing a diaphragm; a first microphone configured to acquire a digital auscultation audio signal through the diaphragm and a second microphone configured to acquire an ambient audio signal; and a housing coupled to the chest piece and enclosing processing circuitry, wherein the processing circuitry is configured to generate and output an active noise cancelation signal based on the ambient audio signal, apply a gain to the digital auscultation audio signal, apply a digital filter to the digital auscultation audio signal, and stream the digital auscultation audio signal to external processing circuitry while acquiring the digital auscultation audio signal, and wherein the external processing circuitry is configured to extract a feature of the digital auscultation audio signal and classify the digital auscultation audio signal as including a Still's murmur based on the extracted feature.

### CROSS-REFERENCE TO RELATED APPLICATIONS

The present application claims priority to U.S. Provisional Application No. 63/496,444, filed Apr. 17, 2023, which is incorporated herein by reference in its entirety for all purposes. Further, the present application is related to U.S. Pat. No. 10,251,562, incorporated herein by reference.

### FEDERAL FUNDING DISCLOSURE

This disclosure was made with government support under Grant Number HL131081 and Grant Number NR019735 each awarded by the National Institutes of Health. The government has certain rights to the disclosure.

### BACKGROUND

### Field of the Disclosure

The present disclosure relates to digital auscultation.

### Description of the Related Art

Auscultation is an essential tool for cardiopulmonary examination. Accurate pediatric auscultation can be especially important for identifying cardiac issues such as Still's murmur.

### SUMMARY

The foregoing paragraphs have been provided by way of general introduction and are not intended to limit the scope of the following claims. The described embodiments, together with further advantages, will be best understood by reference to the following detailed description taken in conjunction with the accompanying drawings.

In one embodiment, the present disclosure is related to a digital stethoscope, comprising: a chest piece enclosing a diaphragm; a first microphone configured to acquire a digital auscultation audio signal through the diaphragm and a second microphone configured to acquire an ambient audio signal; and a housing coupled to the chest piece and enclosing processing circuitry, wherein the processing circuitry is configured to generate and output an active noise cancelation signal based on the ambient audio signal, apply a gain to the digital auscultation audio signal, apply a digital filter to the digital auscultation audio signal, and stream the digital auscultation audio signal to external processing circuitry while the digital auscultation audio signal is acquired by the first microphone, and wherein the external processing circuitry is configured to extract a feature of the digital auscultation audio signal and classify the digital auscultation audio signal as including a Still's murmur based on the extracted feature.

In one embodiment, the present disclosure is related to a digital stethoscope, comprising: a chest piece enclosing a diaphragm; a first microphone configured to acquire a digital auscultation audio signal through the diaphragm and a second microphone configured to acquire an ambient audio signal; and a housing coupled to the chest piece and enclosing processing circuitry, wherein the processing circuitry is configured to generate and output an active noise cancelation signal based on the ambient audio signal, apply a gain to the digital auscultation audio signal, apply a digital filter to the digital auscultation audio signal, and stream the digital auscultation audio signal to external processing circuitry while the digital auscultation audio signal is acquired by the first microphone, and wherein the external processing circuitry is configured to extract a feature of the digital auscultation audio signal and classify the digital auscultation audio signal as including a heart murmur or a type of the heart murmur based on the extracted feature.

In one embodiment, the present disclosure is related to a digital stethoscope, comprising: a chest piece enclosing a diaphragm; a first microphone configured to acquire a digital auscultation audio signal through the diaphragm and a second microphone configured to acquire an ambient audio signal; and a housing coupled to the chest piece and enclosing processing circuitry, wherein the processing circuitry is configured to generate and output an active noise cancelation signal based on the ambient audio signal, apply a gain to the digital auscultation audio signal, apply a digital filter to the digital auscultation audio signal, and stream the digital auscultation audio signal to external processing circuitry while the digital auscultation audio signal is acquired by the first microphone, and wherein the external processing circuitry is configured to detect wheezing based on the digital auscultation audio signal.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1A is a digital stethoscope, according to one embodiment of the present disclosure;

FIG. 1B is a digital stethoscope, according to one embodiment of the present disclosure;

FIG. 1C is a digital stethoscope, according to one embodiment of the present disclosure;

FIG. 1D is a digital stethoscope, according to one embodiment of the present disclosure;

FIG. 1E is a digital stethoscope, according to one embodiment of the present disclosure;

FIG. 2 is an illustration of stethoscope processing circuitry, according to one embodiment of the present disclosure;

FIG. 3 is a schematic of a user device for performing a method, according to one embodiment of the present disclosure;

FIG. 4 is a schematic of a hardware system for performing a method, according to one embodiment of the present disclosure; and

FIG. 5 is a schematic of a hardware configuration of a device for performing a method, according to one embodiment of the present disclosure.

### DETAILED DESCRIPTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

Auscultation can be used to examine internal organs of the body, and specifically the circulatory, respiratory, and gastrointestinal systems, as a diagnostic tool. Stethoscopes are a primary tool for auscultation. Acoustic or analog auscultation requires a trained clinician to listen to the interior of a patient's body and determine whether the patient is healthy. The level of knowledge of healthy and unhealthy (or normal and abnormal) cardiopulmonary sounds that is required to accurately assess a patient via acoustic auscultation presents a barrier to easily accessible medical assessment and diagnosis. In addition, a clinician's ability to assess a patient via acoustic auscultation can be greatly affected by ambient noise in the surrounding environment.

In one embodiment, the present disclosure is directed to systems and methods of digital auscultation. In one embodiment, the present disclosure is directed to a digital stethoscope. The digital stethoscope can receive, record, and process sounds from the interior of a patient's body, including, but not limited to, the heart, lungs, abdomen, back, throat vascular system, and gastrointestinal system. In one embodiment, the audio recorded and processed by the digital stethoscope can be used to automatically generate a diagnosis of a patient. The diagnosis of a patient can include, for example, an identification of Still's murmur based on cardiac data (e.g., using methods such as those disclosed in U.S. Pat. No. 10,251,562), which is a heart murmur that occurs most often in young children, or an identification of wheezing based on pulmonary data (e.g., using methods such as those disclosed in U.S. Pat. No. 11,484,283, incorporated herein by reference). In one embodiment, the digital stethoscope can be designed to record and process audio from pediatric patients. In one embodiment, the digital stethoscope can be used to deliver diagnoses outside of a clinical setting.

In addition, in one embodiment the digital stethoscope can be used to confirm no murmur or an innocent murmur, if a murmur is heard, as part of pre-surgical heart examination. In one embodiment the digital stethoscope can be used for auscultation of abdominal sounds to monitor appropriate placement of gastronomy tube. In one embodiment the digital stethoscope can be used for auscultation to monitor proper functioning of left ventricular assist device as well as other implanted devices. In one embodiment the digital stethoscope can be used for tele-auscultation of heart and lung sounds allowing for converting office visits to telemedicine visits. The digital stethoscope can thus be used for many other indications and uses inside and outside of a clinical setting.

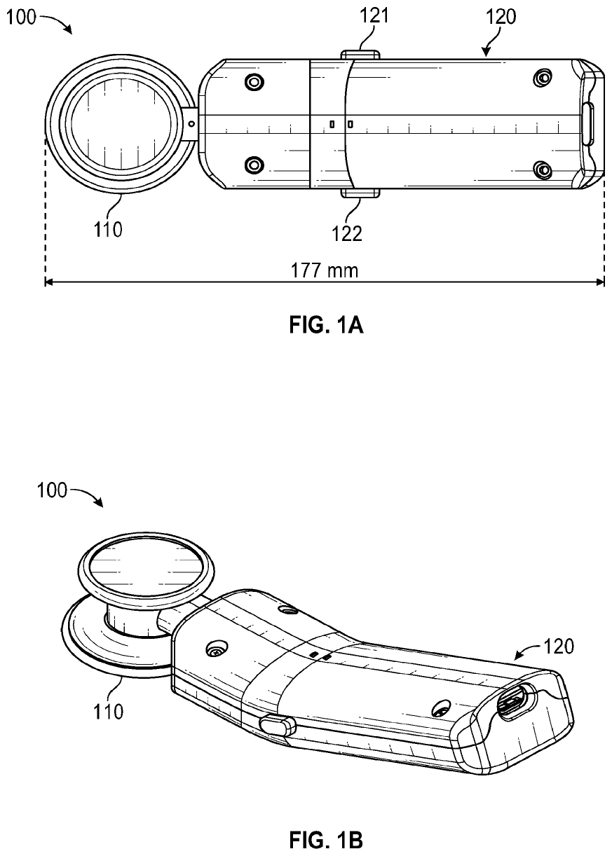

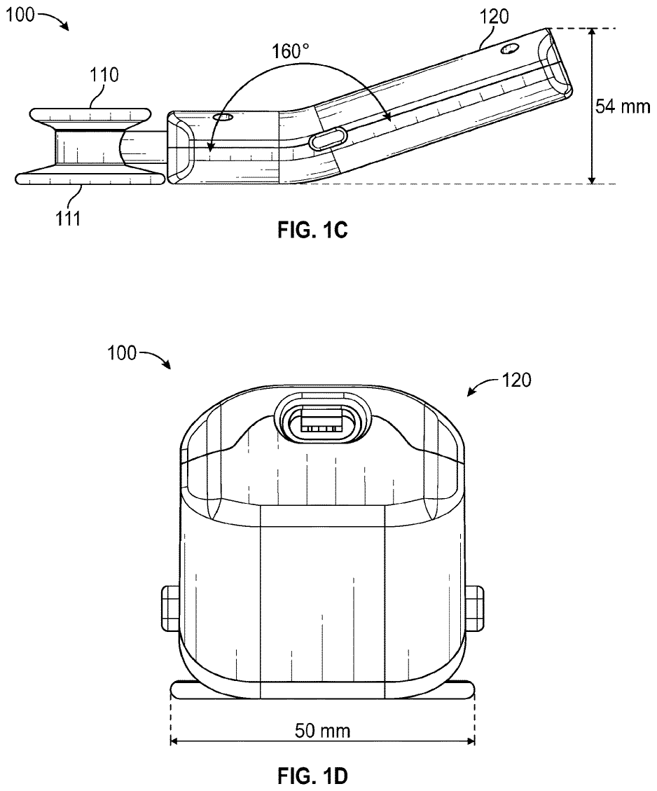

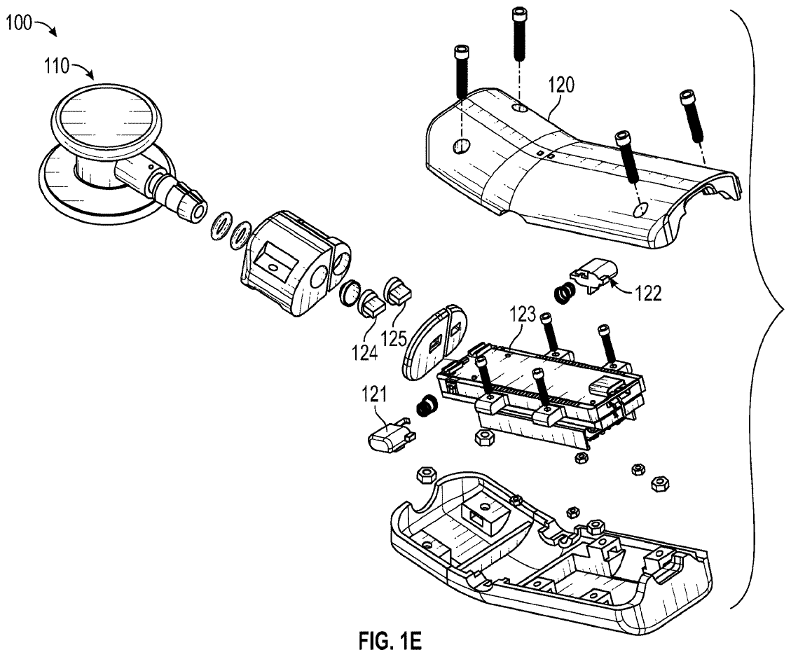

FIG. 1A through FIG. 1E are illustrations of a digital stethoscope 100 according to one embodiment of the present disclosure. FIG. 1A is a front view of the digital stethoscope 100, FIG. 1B is a back view of the digital stethoscope 100, FIG. 1C is a side view of the digital stethoscope 100, and FIG. 1D is a bottom view of the digital stethoscope 100. The digital stethoscope 100 (or electronic stethoscope) can be a stethoscope including a chest piece 110 attached to a body or housing 120. The chest piece 110 can include a diaphragm 111 that can be placed against a patient's skin. The diaphragm 111 can be a tunable diaphragm and can capture a range of frequencies based on a pressure applied to the diaphragm. In one embodiment, the diaphragm 111 can be a two-sided diaphragm such that either surface of the diaphragm 111 can be used to measure and record audio. The diaphragm 111 can measure audio throughout the patient's body, including at the heart, lungs, neck, chest, back, abdomen, gastrointestinal system, and vascular system.

In one embodiment, the body 120 of the digital stethoscope can be an angled body, as illustrated in FIG. 1C. As a non-limiting example, the base of the body 120 can form an angle of approximately 160° with the head of the body where the chest piece 110 is connected to the body. The angle of the body 120 can enable a user to place the diaphragm 111 directly on a patient's skin without the body 120 or the hand of the user interfering with the contact between the diaphragm 111 and the skin. In one embodiment, the body 120 can include a first button 121 on a first side of the body 120 and a second button 122 on an opposing second side of the body 120, as illustrated in FIG. 1A. The first button 121 can be a power button and can be used to turn the digital stethoscope on or off. The second button 122 can be a record button and can be used to initiate recording of an auscultation audio signal through the diaphragm by processing circuitry of the digital stethoscope. The body 120 of the digital stethoscope can include one or more visual indicators, such as lights, to indicate a measurement status or state of the digital stethoscope. The status or state can include, for example, an on/off status, power level, connectivity, charging status, recording status. In one embodiment, the body 120 can include a motion sensor and can be turned on via motion of the digital stethoscope 100.

In one embodiment, the body 120 of the digital stethoscope can be a plastic material. The plastic material can include a plastic filament or resin. In one embodiment, the body 120 of the digital stethoscope can be 3D printed. The design of the body 120 as having two halves that can be coupled together to form a chamber around internal processing circuitry can enable quick and low-cost manufacturing and assembly. For example, the body 120 can be 3D printed in approximately 6 hours or less, and the cost of printing materials can be low (e.g., the cost can be approximately $75 or less).

FIG. 1E is an exploded view of the body 120 of the digital stethoscope according to one embodiment of the present disclosure. The body 120 can enclose stethoscope processing circuitry. In one embodiment, the stethoscope processing circuitry can include a microcontroller mounted on a printed circuit board (PCB) 123. The microcontroller can be configured to receive and record audio signals, process the audio signals, and transmit the audio signals to an external device. In one embodiment, the body 120 of the digital stethoscope can include at least one microphone. The at least one microphone can include a first microphone (primary microphone) 124 and a second microphone (secondary microphone, reference microphone) 125. The first microphone and the second microphone can be in communication with the microcontroller. The microcontroller can transmit instructions to the microphones and can receive data from the microphones. The microcontroller can be communicatively coupled to the microphones via a wired connection or a wireless connection. In one embodiment, the first microphone and the second microphone can be located at the head of the body 120 in proximity to the chest piece 110. The chest piece can form a rigid chamber through which sound can travel to the microphones at the head of the body 120. In one embodiment, the chest piece and body can form a tubeless auscultation system.

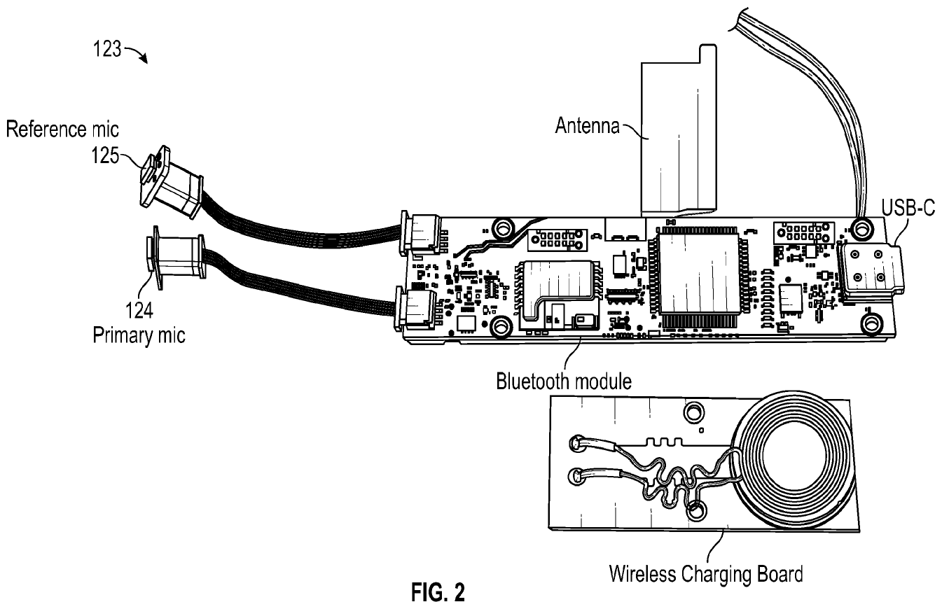

FIG. 2 is an illustration of the PCB 123 according to one embodiment of the present disclosure. In one embodiment, the PCB 123 can include an antenna and a Bluetooth module for wireless data transmission. The primary microphone 124 and the reference microphone 125 can be connected to the microcontroller mounted on the PCB 123 via a wired connection. The microphones can be approximately in line with each other and can be surrounded by a housing. In one embodiment, the housing of the microphones can be designed to reduce noise from friction between components or between the digital stethoscope and the patient. In one embodiment, the PCB 123 can include a rechargeable power source, e.g., a lithium-ion polymer battery. The rechargeable power source can be charged via a wired connection with a charge source. In one embodiment, the rechargeable power source can be wirelessly charged, e.g., via inductive charging of a wireless charging board, as illustrated in FIG. 2.

In one embodiment, the microphones can include a microelectromechanical system (MEMS). A MEMS microphone can be configured to convert sound to electrical signals via an acoustic sensor. The amplitude of an electrical signal (an audio signal) generated by the MEMS microphone can depend on the volume of audio in proximity to the microphone. In one embodiment, a frequency range that can be detected by the digital stethoscope can be between approximately 500 Hz to 1000 Hz. In one embodiment, the frequency range can be between approximately 20 Hz to 2000 Hz. In one embodiment, the microphones can include adjustable gain and/or filters.

In one embodiment, the digital stethoscope 100 can be configured for active noise cancellation (ANC) using the primary microphone and the reference microphone. Auscultation can be performed in environments such as ambulance or hospitals, which can have significant ambient noise. Ambient noise can interfere with the ability of the microphones to detect and record sounds from inside the body. In one embodiment, the primary microphone 124 can record sounds through the chest piece 110. The reference microphone 125 can record ambient or external audio signals from the surroundings of the digital stethoscope. In one embodiment, the stethoscope processing circuitry can generate a noise cancelling signal based on the audio recorded by the reference microphone. The noise cancelling signal can be an audio signal and can be an inverse of an ambient audio signal recorded by at least one of the microphones. The digital stethoscope 100 can then output the noise cancelling signal to the primary microphone to actively cancel ambient audio. Advantageously, the ANC can enable high-quality recording of auscultation audio with a high signal-to-noise ratio (SNR) in a single recording. One or more of the microphones can record auscultation audio signals.

In one embodiment, the microphones can record auscultation audio signals in pulse density modulation (PDM) format at a sampling rate of 2.0476 MHz (over sampling by 256 produces an approximately 8K sample rate). In PDM format, the amplitude of an auscultation audio signal can be encoded in the density of pulses. In one embodiment, an auscultation audio signal in PDM format can be converted to a digital signal format, e.g., pulse code modulation (PCM) format at the 8 k sample rate.

In one embodiment, the digital stethoscope 100 can process an auscultation audio signal acquired by a microphone. The auscultation audio signal can be referred to as a digital auscultation audio signal. The processing can be digital signal processing and can include, but is not limited to, amplification, filtering, smoothing, transforming, etc. In one embodiment, the digital stethoscope 100 can amplify an auscultation audio signal by up to 100 different levels. The degree and control of amplification can be especially useful for pediatric auscultation. In one embodiment, the digital stethoscope 100 can digitally filter an auscultation audio signal. In general, the digital filters can include low-pass filters, high-pass filters, bandpass filters, notch filters, etc. In one embodiment, a digital filter can include a bell filter having a cutoff frequency corresponding to the frequency limit of an analog stethoscope bell and/or a diaphragm filter having a cutoff frequency corresponding to the frequency limit of an analog stethoscope diaphragm. A bell filter can have a lower cutoff frequency than a diaphragm filter. In one embodiment, a digital filter cutoff frequency can be approximately 1 kHz. In one embodiment, a digital filter cutoff frequency can be set based on a type of auscultation audio, e.g., a particular organ or system that is being assessed. The digital filter can remove noise from the auscultation audio signal in PCM format and can result in a high-quality signal. In one embodiment, the auscultation audio signal acquired by the digital stethoscope 100 can be validated using analog auscultation signals. The signals acquired by the digital stethoscope 100 were found to be 98.3% in concurrence with analog signals.

In one embodiment, the digital stethoscope 100 can record an auscultation audio signal. In one embodiment, the processing circuitry of the digital stethoscope 100 can write the auscultation audio signal data to memory. The digital stethoscope 100 can store raw auscultation audio signal data (e.g., prior to digital signal processing) or processed auscultation audio signal data.

In one embodiment, the digital stethoscope 100 can transmit auscultation audio signal data to a user device. The user device can be, for example, a mobile phone, a tablet, a computer, a server, etc. In one embodiment, the digital stethoscope 100 can transmit the auscultation audio signal data via a wireless connection or communication network, such as Bluetooth or WIFI. In one embodiment, the digital stethoscope 100 can be paired with the user device via the wireless connection or communication network. In one embodiment, the digital stethoscope 100 can transmit the auscultation audio signal data to the user device via a wired connection. In one embodiment, the digital stethoscope 100 can stream an auscultation audio signal to the user device. Streaming the auscultation audio signal to the user device in real time can be useful for telehealth and immediate diagnostics. In one embodiment, the user device can request or access the auscultation audio signal data from the digital stethoscope 100 via a web interface or application interface. The user device can be configured to display the auscultation audio signal, process the auscultation audio signal, store the auscultation audio signal, and correlate the auscultation audio signal with patient data. In one embodiment, auscultation audio signals acquired and transmitted to a user device by the digital stethoscope 100 were used to diagnose a patient with pathological and innocent heart murmurs with 98.33% concordance with analog auscultation methods.

In one embodiment, the digital stethoscope 100 can receive instructions related to an auscultation recording protocol from the user device. The instructions can include, for example, an auscultation audio signal acquisition protocol, an amount of gain to apply to an auscultation audio signal, noise cancellation specifications, filter characteristics, etc. In one embodiment, the user device can further process the received auscultation audio signal. For example, the user device can modify the received auscultation audio signal. In one embodiment, the user device can use artificial intelligence (e.g., a machine learning model) to process and characterize the received auscultation audio signal. In one embodiment, the user device can generate and display a visual representation of the auscultation audio signal.

In one embodiment, the auscultation audio signal acquired by the digital stethoscope 100 can be used to assess a patient's health. The digital stethoscope 100 can be used to efficiently acquire large amounts of auscultation audio signal data from different patients due to its ease of use and data transmission capabilities. The acquired auscultation audio signal data can then be used as a library of training data. Algorithms and models can be trained using training signal data from the digital stethoscope 100. The algorithms and models can then be used to characterize and assess test signal data from the same (type of) digital stethoscope 100. The similarities in acquisition protocol and signal processing between the training data and the test data can result in more accurate analysis of the test data. In one embodiment, patient data (e.g., age, sex, health conditions and history, demographic data) can be used in addition to the auscultation audio signal to assess a patient.

In one embodiment, the auscultation audio signal acquired by the digital stethoscope 100 can be used to identify Still's murmur (using, for example, the approach described in U.S. Pat. No. 10,251,562). Still's murmur can be difficult to distinguish from other (non-Still's) heart murmurs via analog auscultation. In general, innocent murmurs, including Still's murmurs, can be difficult to distinguish from pathological murmurs. In one embodiment, the present disclosure is directed to systems and methods for automatically detecting Still's murmur in a digital auscultation audio signal. In one embodiment, a user device can filter, denoise, and segment an auscultation audio signal received from the digital stethoscope 100 using a segmentation algorithm. Each segmented cardiac cycle can be used to generate a spectrogram. The spectrograms can be input to a deep learning model such as a convolutional neural network (CNN). The CNN can be trained to identify Still's murmur using previous auscultation audio signals recorded by a digital stethoscope 100. In one embodiment, the CNN can be trained to identify or classify Still's murmur based on certain signal features and characteristics thereof, such as a location, a signal intensity, an envelope shape, a frequency, a peak width, etc. In one embodiment, the methods described herein can identify Still's murmur with a sensitivity of 91.89%, a specificity of 92.59%, and an accuracy of 92.19% based on a dataset of auscultation audio signals. See e.g., the method discussed in U.S. Pat. No. 10,251,562.

In one embodiment, the digital auscultation audio signal acquired by the digital stethoscope 100 can be used to assess pulmonary function. Auscultation audio signals can be acquired at different positions on the chest to record lung function. For example, a lung scan can involve recording 15-second samples at 11 locations on the anterior, posterior, and sides of the chest. In one embodiment, an auscultation audio signal or a set of auscultation audio signals acquired by a digital stethoscope 100 can be processed to automatically identify wheezing. Wheezing can be characterized by a high-pitched sound emitted during exhalation and/or inhalation. Detection of wheezing can be important for assessing exacerbation of asthma, which is a common chronic pediatric disease. In one embodiment, the digital stethoscope 100 can be used outside of a healthcare setting (e.g., at a user's home) due to its ease of use and wireless connectivity with devices that can provide signal processing and diagnostics. For example, the digital stethoscope 100 can be used for home management of acute asthma.

In one embodiment, the auscultation audio signal acquired by the digital stethoscope 100 can be used to identify wheezing with 79.3% concurrence with an analog auscultation method. In one embodiment, a machine learning model can be used by a user device to assess an auscultation audio signal for pulmonary function. The machine learning model can be a deep learning model. In one embodiment, the machine learning model can be a residual network such as ResNet-18. In one embodiment, the machine learning model can be a harmonic network, which is a type of CNN. The machine learning model can be trained on training data that is recorded by the digital stethoscope 100. In one example, a ResNet-18 model can be used to automatically identify wheezing in an auscultation audio signal from the digital stethoscope with a sensitivity of 77%, a specificity of 70%, and an accuracy of 74%. In one embodiment, a harmonic network can be used to automatically identify wheezing in an auscultation audio signal from the digital stethoscope with a sensitivity of 78%, a specificity of 86%, and an accuracy of 84%.

The digital stethoscope 100 described herein can be used to acquire high-quality auscultation audio signals for patient assessment. The auscultation audio signals can be amplified and filtered in order to isolate audio that corresponds to biological activity. The acquisition of high-quality, amplified auscultation audio is especially important for pediatric medicine. The acquired by the digital stethoscope 100 can be used to automatically identify Still's murmur, pathological murmur, wheezing, and other cardiopulmonary conditions.

Embodiments of the subject matter and the functional operations described in this specification can be implemented by digital electronic circuitry, in tangibly embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of data processing apparatus, such as the stethoscope processing circuitry or the user device. The computer storage medium can be a machine-readable storage device, a machine-readable storage substrate, a random or serial access memory device, or a combination of one or more of them.

Each of the functions of the described embodiments can be implemented by one or more processing circuits/processing circuitry/processing firmware (may also be referred to as a controller). A processing circuit includes a programmed processor (for example, a CPU of FIG. 12), as a processor includes circuitry. A processing circuit can also include devices such as an application specific integrated circuit (ASIC) and circuit components arranged to perform the recited functions.

The term “data processing apparatus” refers to data processing hardware and may encompass all kinds of apparatus, devices, and machines for processing data, including by way of example a programmable processor, a computer, or multiple processors or computers. The apparatus can also be or further include special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, Subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA an ASIC.

Computers suitable for the execution of a computer program include, by way of example, general or special purpose microprocessors or both, or any other kind of central processing unit. Generally, a CPU will receive instructions and data from a read-only memory or a random access memory or both. Elements of a computer are a CPU for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more Such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients (user devices) and servers. A client and server are generally remote from each other and typically interact through a communication network. The relationship of client and server arises by virtue of computer programs running on the respective computers and having a client-server relationship to each other. In an embodiment, a server transmits data, e.g., an HTML page, to a user device, e.g., for purposes of displaying data to and receiving user input from a user interacting with the user device, which acts as a client. Data generated at the user device, e.g., a result of the user interaction, can be received from the user device at the server.

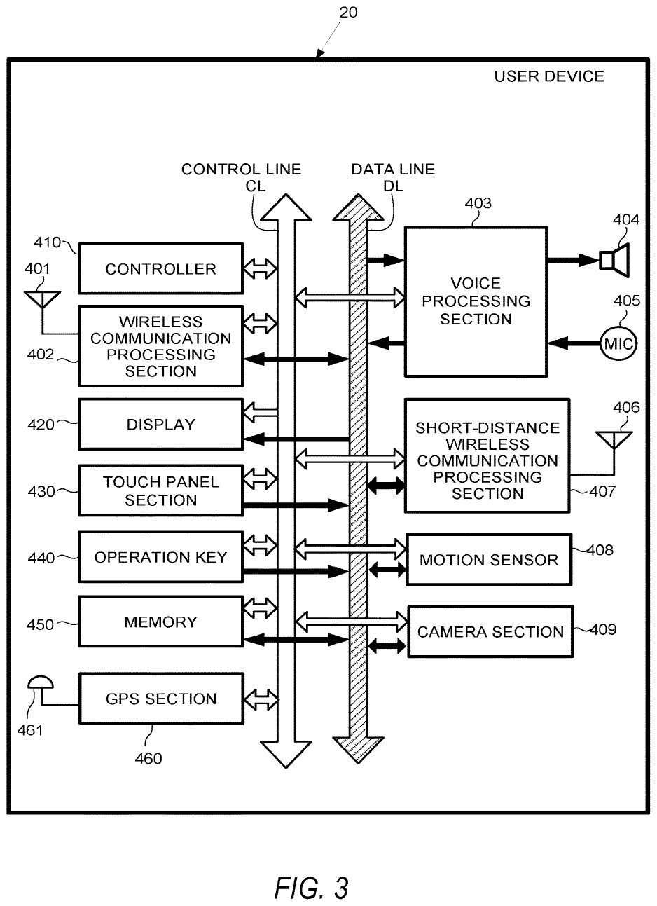

Electronic user device 20 shown in FIG. 3 can be an example of one or more of the devices described herein, including an electronic device configured to receive and process auscultation audio signals and generate a medical diagnosis. In an embodiment, the electronic user device 20 may be a smartphone. However, the skilled artisan will appreciate that the features described herein may be adapted to be implemented on other devices (e.g., a laptop, a tablet, a server, an e-reader, a camera, a navigation device, etc.). In one embodiment, the microprocessor can include components of the user device 20. The user device 20 of FIG. 3 includes processing circuitry, as discussed above. The processing circuitry includes one or more of the elements discussed next with reference to FIG. 3. The electronic user device 20 may include other components not explicitly illustrated in FIG. 3 such as a CPU, GPU, frame buffer, etc. The electronic user device 20 includes a controller 410 and a wireless communication processor 402 connected to an antenna 401. A speaker 404 and a microphone 405 are connected to a voice processor 403.

The controller 410 may include one or more processors/processing circuitry (CPU, GPU, or other circuitry) and may control each element in the user device 20 to perform functions related to communication control, audio signal processing, graphics processing, control for the audio signal processing, still and moving image processing and control, and other kinds of signal processing. The controller 410 may perform these functions by executing instructions stored in a memory 450. Alternatively or in addition to the local storage of the memory 450, the functions may be executed using instructions stored on an external device accessed on a network or on a non-transitory computer readable medium.

The memory 450 includes but is not limited to Read Only Memory (ROM), Random Access Memory (RAM), or a memory array including a combination of volatile and non-volatile memory units. The memory 450 may be utilized as working memory by the controller 410 while executing the processes and algorithms of the present disclosure. Additionally, the memory 450 may be used for long-term storage, e.g., of image data and information related thereto.

The user device 20 includes a control line CL and data line DL as internal communication bus lines. Control data to/from the controller 410 may be transmitted through the control line CL. The data line DL may be used for transmission of voice data, displayed data, etc.

The antenna 401 transmits/receives electromagnetic wave signals between base stations for performing radio-based communication, such as the various forms of cellular telephone communication. The wireless communication processor 402 controls the communication performed between the user device 20 and other external devices via the antenna 401. For example, the wireless communication processor 402 may control communication between base stations for cellular phone communication.

The speaker 404 emits an audio signal corresponding to audio data supplied from the voice processor 403. The microphone 405 detects surrounding audio and converts the detected audio into an audio signal. The audio signal may then be output to the voice processor 403 for further processing. The voice processor 403 demodulates and/or decodes the audio data read from the memory 450 or audio data received by the wireless communication processor 402 and/or a short-distance wireless communication processor 407. Additionally, the voice processor 403 may decode audio signals obtained by the microphone 405.

The user device 20 may also include a display 420, a touch panel 430, an operation key 440, and a short-distance communication processor 407 connected to an antenna 406. The display 420 may be a Liquid Crystal Display (LCD), an organic electroluminescence display panel, or another display screen technology. In addition to displaying still and moving image data, the display 420 may display operational inputs, such as numbers or icons which may be used for control of the user device 20. The display 420 may additionally display a GUI for a user to control aspects of the user device 20 and/or other devices. Further, the display 420 may display characters and images received by the user device 20 and/or stored in the memory 450 or accessed from an external device on a network. For example, the user device 20 may access a network such as the Internet and display text and/or images transmitted from a Web server.

The touch panel 430 may include a physical touch panel display screen and a touch panel driver. The touch panel 430 may include one or more touch sensors for detecting an input operation on an operation surface of the touch panel display screen. The touch panel 430 also detects a touch shape and a touch area. Used herein, the phrase “touch operation” refers to an input operation performed by touching an operation surface of the touch panel display with an instruction object, such as a finger, thumb, or stylus-type instrument. In the case where a stylus or the like is used in a touch operation, the stylus may include a conductive material at least at the tip of the stylus such that the sensors included in the touch panel 430 may detect when the stylus approaches/contacts the operation surface of the touch panel display (similar to the case in which a finger is used for the touch operation).

In certain aspects of the present disclosure, the touch panel 430 may be disposed adjacent to the display 420 (e.g., laminated) or may be formed integrally with the display 420. For simplicity, the present disclosure assumes the touch panel 430 is formed integrally with the display 420 and therefore, examples discussed herein may describe touch operations being performed on the surface of the display 420 rather than the touch panel 430. However, the skilled artisan will appreciate that this is not limiting.

For simplicity, the present disclosure assumes the touch panel 430 is a capacitance-type touch panel technology. However, it should be appreciated that aspects of the present disclosure may easily be applied to other touch panel types (e.g., resistance-type touch panels) with alternate structures. In certain aspects of the present disclosure, the touch panel 430 may include transparent electrode touch sensors arranged in the X-Y direction on the surface of transparent sensor glass.

The touch panel driver may be included in the touch panel 430 for control processing related to the touch panel 430, such as scanning control. For example, the touch panel driver may scan each sensor in an electrostatic capacitance transparent electrode pattern in the X direction and Y-direction and detect the electrostatic capacitance value of each sensor to determine when a touch operation is performed. The touch panel driver may output a coordinate and corresponding electrostatic capacitance value for each sensor. The touch panel driver may also output a sensor identifier that may be mapped to a coordinate on the touch panel display screen. Additionally, the touch panel driver and touch panel sensors may detect when an instruction object, such as a finger is within a predetermined distance from an operation surface of the touch panel display screen. That is, the instruction object does not necessarily need to directly contact the operation surface of the touch panel display screen for touch sensors to detect the instruction object and perform processing described herein. For example, in an embodiment, the touch panel 430 may detect a position of a user's finger around an edge of the display panel 420 (e.g., gripping a protective case that surrounds the display/touch panel). Signals may be transmitted by the touch panel driver, e.g., in response to a detection of a touch operation, in response to a query from another element based on timed data exchange, etc.

The touch panel 430 and the display 420 may be surrounded by a protective casing, which may also enclose the other elements included in the user device 20. In an embodiment, a position of the user's fingers on the protective casing (but not directly on the surface of the display 420) may be detected by the touch panel 430 sensors. Accordingly, the controller 410 may perform display control processing described herein based on the detected position of the user's fingers gripping the casing. For example, an element in an interface may be moved to a new location within the interface (e.g., closer to one or more of the fingers) based on the detected finger position.

Further, in an embodiment, the controller 410 may be configured to detect which hand is holding the user device 20, based on the detected finger position. For example, the touch panel 430 sensors may detect fingers on the left side of the user device 20 (e.g., on an edge of the display 420 or on the protective casing), and detect a single finger on the right side of the user device 20. In this example, the controller 410 may determine that the user is holding the user device 20 with his/her right hand because the detected grip pattern corresponds to an expected pattern when the user device 20 is held only with the right hand.

The operation key 440 may include one or more buttons or similar external control elements, which may generate an operation signal based on a detected input by the user. In addition to outputs from the touch panel 430, these operation signals may be supplied to the controller 410 for performing related processing and control. In certain aspects of the present disclosure, the processing and/or functions associated with external buttons and the like may be performed by the controller 410 in response to an input operation on the touch panel 430 display screen rather than the external button, key, etc. In this way, external buttons on the user device 20 may be eliminated in lieu of performing inputs via touch operations, thereby improving watertightness.

The antenna 406 may transmit/receive electromagnetic wave signals to/from other external apparatuses, and the short-distance wireless communication processor 407 may control the wireless communication performed between the other external apparatuses. Bluetooth, IEEE 802.11, and near-field communication (NFC) are non-limiting examples of wireless communication protocols that may be used for inter-device communication via the short-distance wireless communication processor 407.

The user device 20 may include a motion sensor 408. The motion sensor 408 may detect features of motion (i.e., one or more movements) of the user device 20. For example, the motion sensor 408 may include an accelerometer to detect acceleration, a gyroscope to detect angular velocity, a geomagnetic sensor to detect direction, a geo-location sensor to detect location, etc., or a combination thereof to detect motion of the user device 20. In an embodiment, the motion sensor 408 may generate a detection signal that includes data representing the detected motion. For example, the motion sensor 408 may determine a number of distinct movements in a motion (e.g., from start of the series of movements to the stop, within a predetermined time interval, etc.), a number of physical shocks on the user device 20 (e.g., a jarring, hitting, etc., of the electronic device), a speed and/or acceleration of the motion (instantaneous and/or temporal), or other motion features. The detected motion features may be included in the generated detection signal. The detection signal may be transmitted, e.g., to the controller 410, whereby further processing may be performed based on data included in the detection signal. The motion sensor 408 can work in conjunction with a Global Positioning System (GPS) section 460. The information of the present position detected by the GPS section 460 is transmitted to the controller 410. An antenna 461 is connected to the GPS section 460 for receiving and transmitting signals to and from a GPS satellite.

The user device 20 may include a camera section 409, which includes a lens and shutter for capturing photographs of the surroundings around the user device 20. In an embodiment, the camera section 409 captures surroundings of an opposite side of the user device 20 from the user. The images of the captured photographs can be displayed on the display panel 420. A memory section saves the captured photographs. The memory section may reside within the camera section 109 or it may be part of the memory 450. The camera section 409 can be a separate feature attached to the user device 20 or it can be a built-in camera feature.

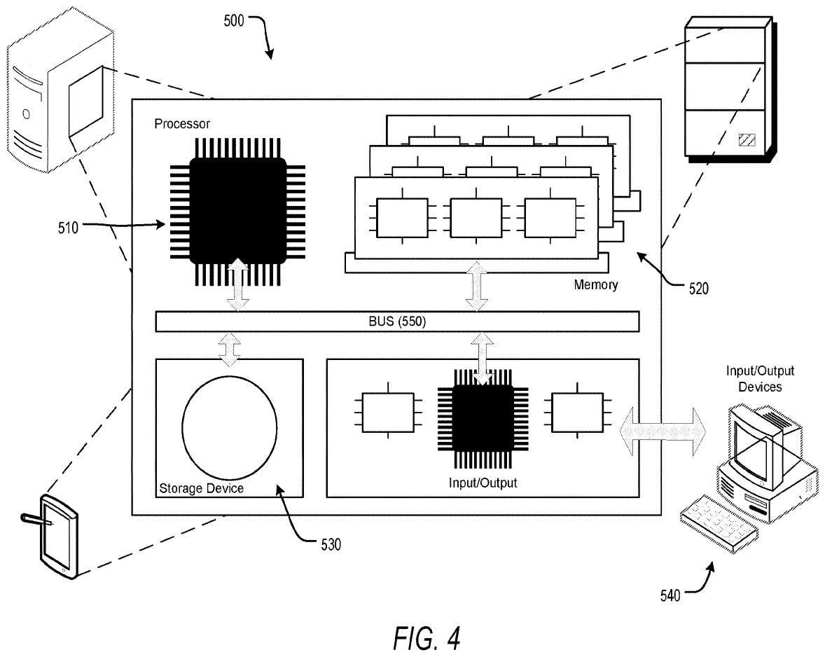

An example of a type of computer is shown in FIG. 4. The computer 500 can be used for the operations described in association with any of the computer-implement methods described previously, according to one implementation. For example, the computer 500 can be an example of an electronic device, such as a computer or mobile device, or a networked device such as a server. The processing circuitry includes one or more of the elements discussed next with reference to FIG. 4. In FIG. 4, the computer 500 includes a processor 510, a memory 520, a storage device 530, and an input/output device 540. Each of the components 510, 520, 530, and 540 are interconnected using a system bus 550. The processor 510 is capable of processing instructions for execution within the system 500. In one implementation, the processor 510 is a single-threaded processor. In another implementation, the processor 510 is a multi-threaded processor. The processor 510 is capable of processing instructions stored in the memory 520 or on the storage device 530 to display graphical information for a user interface on the input/output device 540.

The memory 520 stores information within the computer 500. In one implementation, the memory 520 is a computer-readable medium. In one implementation, the memory 520 is a volatile memory unit. In another implementation, the memory 520 is a non-volatile memory unit.

The storage device 530 is capable of providing mass storage for the computer 500. In one implementation, the storage device 530 is a computer-readable medium. In various different implementations, the storage device 530 may be a floppy disk device, a hard disk device, an optical disk device, or a tape device.

The input/output device 540 provides input/output operations for the computer 500. In one implementation, the input/output device 540 includes a keyboard and/or pointing device. In another implementation, the input/output device 540 includes a display unit for displaying graphical user interfaces.

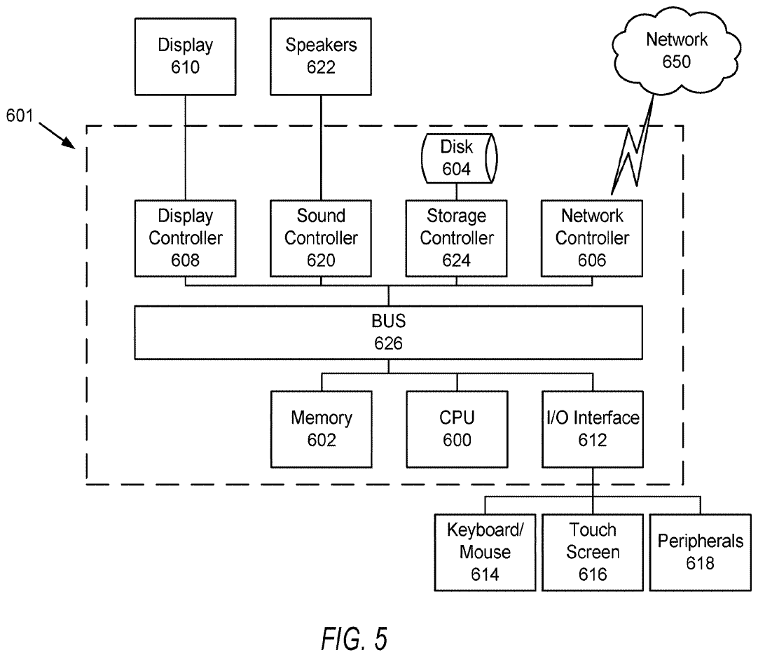

Next, a hardware description of a device 601 according to the present embodiments is described with reference to FIG. 5. In FIG. 5, the device 601, which can be any of the above described devices, including the electronic devices and the networked devices, includes processing circuitry. The processing circuitry includes one or more of the elements discussed next with reference to FIG. 5. The process data and instructions may be stored in memory 602. These processes and instructions may also be stored on a storage medium disk 604 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the device 601 communicates, such as a server or computer.

Further, the claimed advancements may be provided as a utility application, background daemon, or component of an operating system, or combination thereof, executing in conjunction with CPU 600 and an operating system such as Microsoft Windows, UNIX, Solaris, LINUX, Apple MAC-OS and other systems known to those skilled in the art.

The hardware elements in order to achieve the device 601 may be realized by various circuitry elements, known to those skilled in the art. For example, CPU 600 may be a Xenon or Core processor from Intel of America or an Opteron processor from AMD of America, or may be other processor types that would be recognized by one of ordinary skill in the art. Alternatively, the CPU 600 may be implemented on an FPGA, ASIC, PLD or using discrete logic circuits, as one of ordinary skill in the art would recognize. Further, CPU 600 may be implemented as multiple processors cooperatively working in parallel to perform the instructions of the processes described above.

The device 601 in FIG. 5 also includes a network controller 606, such as an Intel Ethernet PRO network interface card from Intel Corporation of America, for interfacing with network 650. and to communicate with the other devices. As can be appreciated, the network 650 can be a public network, such as the Internet, or a private network such as an LAN or WAN network, or any combination thereof and can also include PSTN or ISDN sub-networks. The network 650 can also be wired, such as an Ethernet network, or can be wireless such as a cellular network including EDGE, 3G, 4G and 5G wireless cellular systems. The wireless network can also be WiFi, Bluetooth, or any other wireless form of communication that is known.

The device 601 further includes a display controller 608, such as a NVIDIA Geforce GTX or Quadro graphics adaptor from NVIDIA Corporation of America for interfacing with display 610, such as an LCD monitor. A general purpose I/O interface 612 interfaces with a keyboard and/or mouse 614 as well as a touch screen panel 616 on or separate from display 610. General purpose I/O interface also connects to a variety of peripherals 618 including printers and scanners.

A sound controller 620 is also provided in the device 601 to interface with speakers/microphone 622 thereby providing sounds and/or music.

The general purpose storage controller 624 connects the storage medium disk 604 with communication bus 626, which may be an ISA, EISA, VESA, PCI, or similar, for interconnecting all of the components of the device 601. A description of the general features and functionality of the display 610, keyboard and/or mouse 614, as well as the display controller 608, storage controller 624, network controller 606, sound controller 620, and general purpose I/O interface 612 is omitted herein for brevity as these features are known.

While this specification contains many specific implementation details, these should not be construed as limitations on the scope of what may be claimed, but rather as descriptions of features that may be specific to particular embodiments.

Certain features that are described in this specification in the context of separate embodiments can also be implemented in combination in a single embodiment. Conversely, various features that are described in the context of a single embodiment can also be implemented in multiple embodiments separately or in any suitable sub-combination.

Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system modules and components in the embodiments described above should not be understood as requiring such separation in all embodiments, and it should be understood that the described program components and systems can generally be integrated together in a single component or packaged into multiple components.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

Obviously, numerous modifications and variations are possible in light of the above teachings. It is therefore to be understood that within the scope of the appended claims, embodiments of the present disclosure may be practiced otherwise than as specifically described herein.

Embodiments of the present disclosure may also be as set forth in the following parentheticals:
(1) A digital stethoscope, comprising a chest piece enclosing a diaphragm; a first microphone configured to acquire a digital auscultation audio signal through the diaphragm and a second microphone configured to acquire an ambient audio signal; and a housing coupled to the chest piece and enclosing processing circuitry, wherein the processing circuitry is configured to: generate and output an active noise cancelation signal based on the ambient audio signal, apply a gain to the digital auscultation audio signal, apply a digital filter to the digital auscultation audio signal, and stream the digital auscultation audio signal to external processing circuitry while the digital auscultation audio signal is acquired by the first microphone, and wherein the external processing circuitry is configured to extract a feature of the digital auscultation audio signal and classify the digital auscultation audio signal as including a Still's murmur based on the extracted feature.
(2) The digital stethoscope of (1), wherein the digital filter is a low-pass filter having a cutoff frequency of 1 kHz.
(3) The digital stethoscope of (1) to (2), wherein the digital filter has a cutoff frequency corresponding to a frequency range of a stethoscope bell or a stethoscope diaphragm.
(4) The digital stethoscope of (1) to (3), wherein the external processing circuitry is configured to classify the digital auscultation audio signal using a convolutional neural network (CNN).
(5) The digital stethoscope of (1) to (4), wherein the processing circuitry is configured to stream the digital auscultation audio signal to the external processing circuitry via a Bluetooth connection.
(6) The digital stethoscope of (1) to (5), wherein the processing circuitry is configured to receive a gain amount or a digital filter cutoff frequency from the external processing circuitry.
(7) A digital stethoscope, comprising: a chest piece enclosing a diaphragm; a first microphone configured to acquire a digital auscultation audio signal through the diaphragm and a second microphone configured to acquire an ambient audio signal; and a housing coupled to the chest piece and enclosing processing circuitry, wherein the processing circuitry is configured to: generate and output an active noise cancelation signal based on the ambient audio signal, apply a gain to the digital auscultation audio signal, apply a digital filter to the digital auscultation audio signal, and stream the digital auscultation audio signal to external processing circuitry while the digital auscultation audio signal is acquired by the first microphone, and wherein the external processing circuitry is configured to extract a feature of the digital auscultation audio signal and classify the digital auscultation audio signal as including a heart murmur based on the extracted feature.
(8) The digital stethoscope of (7), wherein the digital filter is a low-pass filter having a cutoff frequency of 1 kHz.
(9) The digital stethoscope of (7) to (8), wherein the digital filter has a cutoff frequency corresponding to a frequency range of a stethoscope bell or a stethoscope diaphragm.
(10) The digital stethoscope of (7) to (9), wherein the external processing circuitry is configured to classify the digital auscultation audio signal using a convolutional neural network (CNN).
(11) The digital stethoscope of (7) to (10), wherein the processing circuitry is configured to stream the digital auscultation audio signal to the external processing circuitry via a Bluetooth connection.
(12) The digital stethoscope of (7) to (11), wherein the processing circuitry is configured to receive a gain amount or a digital filter cutoff frequency from the external processing circuitry.
(13) The digital stethoscope of (7) to (12), wherein the external processing circuitry is configured to classify the digital auscultation audio signal as including a pathological heart murmur or an innocent heart murmur.
(14) A digital stethoscope, comprising: a chest piece enclosing a diaphragm; a first microphone configured to acquire a digital auscultation audio signal through the diaphragm and a second microphone configured to acquire an ambient audio signal; and a housing coupled to the chest piece and enclosing processing circuitry, wherein the processing circuitry is configured to: generate and output an active noise cancelation signal based on the ambient audio signal, apply a gain to the digital auscultation audio signal, apply a digital filter to the digital auscultation audio signal, and stream the digital auscultation audio signal to external processing circuitry while the digital auscultation audio signal is acquired by the first microphone, and wherein the external processing circuitry is configured to detect wheezing based on the digital auscultation audio signal.
(15) The digital stethoscope of (14), wherein the digital filter is a low-pass filter having a cutoff frequency of 1 kHz.
(16) The digital stethoscope of (14) to (15), wherein the digital filter has a cutoff frequency corresponding to a frequency range of a stethoscope bell or a stethoscope diaphragm.
(17) The digital stethoscope of (14) to (16), wherein the external processing circuitry is configured to detect wheezing using a residual neural network or a harmonic network.
(18) The digital stethoscope of (14) to (17), wherein the external processing circuitry is configured to detect wheezing based on the digital auscultation audio signal using a deep learning model.
(19) The digital stethoscope of (14) to (18), wherein the processing circuitry is configured to stream the digital auscultation audio signal to the external processing circuitry via a Bluetooth connection.
(20) The digital stethoscope of (14) to (19), wherein the processing circuitry is configured to receive a gain amount or a digital filter cutoff frequency from the external processing circuitry.

Thus, the foregoing discussion discloses and describes merely exemplary embodiments of the present disclosure. As will be understood by those skilled in the art, the present disclosure may be embodied in other specific forms without departing from the spirit thereof. Accordingly, the disclosure of the present disclosure is intended to be illustrative, but not limiting of the scope of the disclosure, as well as other claims. The disclosure, including any readily discernible variants of the teachings herein, defines, in part, the scope of the foregoing claim terminology such that no inventive subject matter is dedicated to the public.

1. A digital stethoscope, comprising:
a chest piece enclosing a diaphragm;
a first microphone configured to acquire a digital auscultation audio signal through the diaphragm and a second microphone configured to acquire an ambient audio signal; and
a housing coupled to the chest piece and enclosing processing circuitry,
wherein the processing circuitry is configured to:
generate and output an active noise cancelation signal based on the ambient audio signal,
apply a gain to the digital auscultation audio signal,
apply a digital filter to the digital auscultation audio signal, and
stream the digital auscultation audio signal to external processing circuitry while the digital auscultation audio signal is acquired by the first microphone, and
wherein the external processing circuitry is configured to extract a feature of the digital auscultation audio signal and classify the digital auscultation audio signal as including a Still's murmur based on the extracted feature.

2. The digital stethoscope of claim 1, wherein the digital filter is a low-pass filter having a cutoff frequency of 1 kHz.

3. The digital stethoscope of claim 1, wherein the digital filter has a cutoff frequency corresponding to a frequency range of a stethoscope bell or a stethoscope diaphragm.

4. The digital stethoscope of claim 1, wherein the external processing circuitry is configured to classify the digital auscultation audio signal using a deep learning model.

5. The digital stethoscope of claim 1, wherein the processing circuitry is configured to stream the digital auscultation audio signal to the external processing circuitry via a Bluetooth connection.

6. The digital stethoscope of claim 1, wherein the processing circuitry is configured to receive a gain amount or a digital filter cutoff frequency from the external processing circuitry.

7. A digital stethoscope, comprising:
a chest piece enclosing a diaphragm;
a first microphone configured to acquire a digital auscultation audio signal through the diaphragm and a second microphone configured to acquire an ambient audio signal; and
a housing coupled to the chest piece and enclosing processing circuitry,
wherein the processing circuitry is configured to:
generate and output an active noise cancelation signal based on the ambient audio signal,
apply a gain to the digital auscultation audio signal,
apply a digital filter to the digital auscultation audio signal, and
stream the digital auscultation audio signal to external processing circuitry while the digital auscultation audio signal is acquired by the first microphone, and
wherein the external processing circuitry is configured to extract a feature of the digital auscultation audio signal and classify the digital auscultation audio signal as including a heart murmur based on the extracted feature.

8. The digital stethoscope of claim 7, wherein the digital filter is a low-pass filter having a cutoff frequency of 1 kHz.

9. The digital stethoscope of claim 7, wherein the digital filter has a cutoff frequency corresponding to a frequency range of a stethoscope bell or a stethoscope diaphragm.

10. The digital stethoscope of claim 7, wherein the external processing circuitry is configured to classify the digital auscultation audio signal using a deep learning model.

11. The digital stethoscope of claim 7, wherein the processing circuitry is configured to stream the digital auscultation audio signal to the external processing circuitry via a Bluetooth connection.

12. The digital stethoscope of claim 7, wherein the processing circuitry is configured to receive a gain amount or a digital filter cutoff frequency from the external processing circuitry.

13. The digital stethoscope of claim 7, wherein the external processing circuitry is configured to classify the digital auscultation audio signal as including a pathological heart murmur or an innocent heart murmur.

14. A digital stethoscope, comprising:
a chest piece enclosing a diaphragm;
a first microphone configured to acquire a digital auscultation audio signal through the diaphragm and a second microphone configured to acquire an ambient audio signal; and
a housing coupled to the chest piece and enclosing processing circuitry,
wherein the processing circuitry is configured to:
generate and output an active noise cancelation signal based on the ambient audio signal,
apply a gain to the digital auscultation audio signal,
apply a digital filter to the digital auscultation audio signal, and
stream the digital auscultation audio signal to external processing circuitry while the digital auscultation audio signal is acquired by the first microphone, and
wherein the external processing circuitry is configured to detect wheezing based on the digital auscultation audio signal.

15. The digital stethoscope of claim 14, wherein the digital filter is a low-pass filter having a cutoff frequency of 1 kHz.

16. The digital stethoscope of claim 14, wherein the digital filter has a cutoff frequency corresponding to a frequency range of a stethoscope bell or a stethoscope diaphragm.

17. The digital stethoscope of claim 14, wherein the external processing circuitry is configured to detect wheezing using a residual neural network or a harmonic network.

18. The digital stethoscope of claim 14, wherein the external processing circuitry is configured to detect wheezing based on the digital auscultation audio signal using a deep learning model.

19. The digital stethoscope of claim 14, wherein the processing circuitry is configured to stream the digital auscultation audio signal to the external processing circuitry via a Bluetooth connection.

20. The digital stethoscope of claim 14, wherein the processing circuitry is configured to receive a gain amount or a digital filter cutoff frequency from the external processing circuitry.
